(19) United States
(12) Patent Application Publication
(10) Pub. No.: US 2011/0137375 A1
(43) Pub. Date: Jun. 9, 2011

(54) SYSTEM AND METHOD FOR DETECTION OF INVERSION AND EVERSION OF THE FOOT USING A MULTI-CHAMBER INSOLE

(76) Inventor: Keith MCBRIDE, Ventura, CA (US)

(21) Appl. No.: 12/630,199

(22) Filed: Dec. 3, 2009

(51) Int. Cl. A61N 1/36

(52) U.S. Cl. 607/49; 607/48

(57) ABSTRACT

A system includes at least three sensors, an electrode array, a muscle stimulator, and a microprocessor. The sensors are configured to be arranged substantially in a common plane and associated with a foot of a body. Additionally, each of the sensors are configured to produce a signal associated with an orientation of the foot. The electrode array is coupled to a lower limb of the body and configured to stimulate at least one muscle of the lower limb. The muscle stimulator is coupled to the electrode array and configured to output a muscle stimulation signal to the electrode array. The microprocessor is coupled to the sensors and configured to calculate an orientation of the foot based on the signals produced by the sensors. Additionally, the microprocessor is configured to control the muscle stimulation output via the muscle stimulator based on the calculated orientation of the foot.

### CROSS-REFERENCE TO RELATED APPLICATIONS

This application is related to U.S. patent application Ser. No. 10/335,905, entitled “Neuroprosthesis System for Improved Lower-Limb Function,” filed Jan. 3, 2003, which is incorporated herein by reference in its entirety. This application is related to U.S. patent application Ser. No. 11/380,430, entitled “Orthosis for a Gait Modulation System,” filed Apr. 27, 2006, which is incorporated herein by reference in its entirety. This application is related to U.S. patent application Ser. No. 11/552,997, entitled “Sensor Device for Gait Enhancement,” filed Oct. 26, 2006, which is incorporated herein by reference in its entirety.

### BACKGROUND

The invention relates generally to a medical device and method for functional electrical stimulation (FES) of lower limbs, and more particularly to a medical device and method for improving lower limb function of users having neuromuscular impairment of the lower limbs.

It is known that pathologies of the neuromuscular system due to disease or trauma to the central nervous system, such as, for example, a stroke, spinal cord injury, head injury, cerebral palsy and multiple sclerosis, can impede walking and leg function. In some instances, as a result of injury, the electrical paths from the brain to the lower extremities become damaged such that the muscles and sensors in the region become paralyzed. Leg muscles, however, can generally be activated with Functional Electrical Stimulation (FES), a methodology that uses bursts of short electrical pulses, applied to motor nerves, to generate muscle contraction.

One of the most common neurological impairments associated with lower limb function is dropfoot (or foot drop). Foot drop can result in insufficient lifting of the distal foot segment on the affected side of the body during the swinging phase of the gait cycle. Moreover, foot drop can result in inversion or eversion of the foot during the swinging phase of the gait cycle. Currently, detection of foot drop and correction using FES is limited and does not include the detection of inversion or eversion of the foot, as described above.

Thus, a need exists for a system and method for detection of inversion and eversion of the foot and associated systems to correct such issues.

### SUMMARY

Systems and methods for detecting and correcting foot drop are described herein. In some embodiments, a system includes at least three sensors, an electrode array, a muscle stimulator, and a microprocessor. The sensors are configured to be arranged substantially in a common plane and associated with a foot of a body. Additionally, each of the sensors are configured to produce a signal associated with an orientation of the foot. The electrode array is coupled to a lower limb of the body and configured to stimulate at least one muscle of the lower limb. The muscle stimulator is coupled to the electrode array and configured to output a muscle stimulation signal to the electrode array. The microprocessor is coupled to the sensors and configured to calculate an orientation of the foot based on the signals produced by the sensors. Additionally, the microprocessor is configured to control the muscle stimulation output via the muscle stimulator based on the calculated orientation of the foot.

### BRIEF DESCRIPTION OF THE DRAWINGS

FIG. 1 is a schematic illustration of a system according to an embodiment.

FIG. 2 is a perspective view of a system according to an embodiment.

FIG. 3 is a block diagram of a stimulator unit according to an embodiment.

FIG. 4 is a perspective view of an orthosis according to an embodiment.

FIGS. 5a-5c are schematic illustrations of sensor configurations according to embodiments.

FIG. 6 is a schematic illustration of a sensor configuration according to an embodiment.

FIG. 7 is a flow chart of a method according to an embodiment.

### DETAILED DESCRIPTION

In some embodiments, a system includes at least three sensors, an electrode array, a muscle stimulator, and a microprocessor. The sensors are configured to be arranged substantially in a common plane and associated with a foot of a body. Additionally, each of the sensors are configured to produce a signal associated with an orientation of the foot. The electrode array is coupled to a lower limb of the body and configured to stimulate at least one muscle of the lower limb. The muscle stimulator is coupled to the electrode array and configured to output a muscle stimulation signal to the electrode array. The microprocessor is coupled to the sensors and configured to calculate an orientation of the foot based on the signals produced by the sensors. Additionally, the microprocessor is configured to control the muscle stimulation output via the muscle stimulator based on the calculated orientation of the foot.

In some embodiments, a method includes receiving data from at least three sensors associated with a foot of a body. The orientation of the foot is calculated with respect to a lower limb of the body based on the data received from the sensors. The stimulation of at least one muscle of the lower limb is determined based on the calculated orientation of the foot. The stimulation output to the at least one muscle of the lower limb via a muscle stimulator is controlled. In some embodiments, a processor is instructed via code to receive data, calculate the orientation of the foot, determine at least one muscle to stimulate, and control the stimulation output.

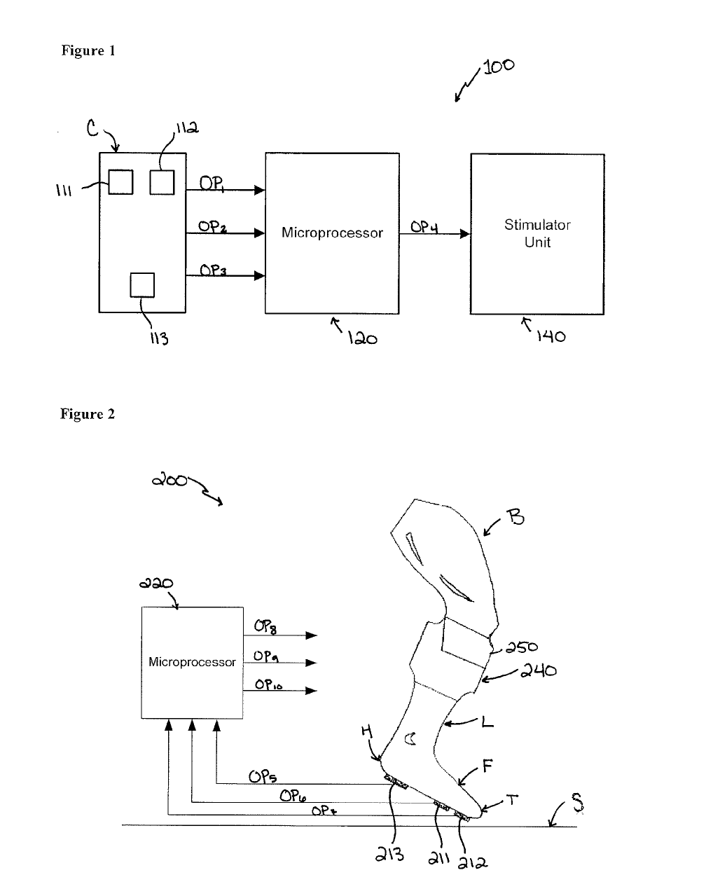

FIG. 1 is a schematic illustration of a system 100 according to an embodiment of the invention. The system 100 is configured to detect and correct an orientation of a foot of a body, such as, for example, an inversion or an eversion, during a gait cycle. The system 100 includes three sensors 111, 112, and 113, a microprocessor 120, and a stimulator unit 140. The sensors 111, 112, and 113 are arranged substantially in a common plane C and are associated with a foot of a body. The sensors 111, 112, and 113 can be, for example, one of an on-off switch, a force sensor, a pressure sensor, a gyroscopic tilt sensor, an accelerometer, a goniometer and/or the like. The common plane can be, for example, associated with the bottom of the foot such that the sensors 111, 112, and 113 are coupled to the bottom of the foot. In other embodiments, the common plane can be associated with a sole of a shoe such that the sensors 111, 112, and 113 are placed within the sole of the shoe.

The sensors 111, 112, and 113 are configured to facilitate the detection of the orientation of the foot. As such, the sensors 111, 112, and 113 can be arranged in any configuration in the common plane C that facilitates the detection of the orientation of the foot, as discussed in more detail herein. The sensors 111, 112, and 113 are each configured to produce output signals OP₁, OP₂, and OP₃, respectively, associated with the orientation of the foot. In some embodiments, the output signals OP₁, OP₂, and OP₃ of the sensors 111, 112, and 113 are associated with a threshold value. For example, in some embodiments, Boolean logic signals are used such that thresholds can be pre-determined force thresholds (e.g., Threshold₁ and Threshold₂) designated by 0. In some such embodiments, when Threshold₁ and Threshold₂ are exceeded they are designated by 1.

The sensors 111, 112, and 113 are coupled to the microprocessor 120 such that the microprocessor 120 receives the output signals OP₁, OP₂, and OP₃, generated by the sensors 111, 112, and 113. The microprocessor 120 can be, for example, an application-specific integrated circuit (ASIC) or a combination of ASICs, which are designed to perform one or more specific functions. In some embodiments, the microprocessor 120 can be an analog or digital circuit, or a combination of multiple circuits. The microprocessor 120 is configured to calculate the orientation of the foot based on the output signals OP₁, OP₂, and OP₃, of the sensors 111, 112 and 113. In some embodiments, the microprocessor 120 can calculate the orientation of the foot using the relative position of each of the sensors 111, 112 and 113 on the common plane C in combination with the output signals OP₁, OP₂, and OP₃, of the sensors 111, 112 and 113. In some embodiments, the microprocessor 120 can calculate the orientation of the foot by comparing the output signals OP₁, OP₂, and OP₃ of the sensors 111, 112 and 113 with a threshold value associated with each of the sensors 111, 112 and 113. In this manner, the microprocessor 120 can determine, for example, whether the foot is in an inverted or an everted orientation during a gait cycle. In some embodiments, the microprocessor 120 can further calculate the position of the foot along the path of the gait cycle.

When the microprocessor 120 calculates an abnormal orientation of the foot during a gait cycle, such as, for example, an inverted or everted orientation, the microprocessor 120 is configured to determine the muscle attributable to the abnormal orientation. For example, the microprocessor 120 could determine that a tibialis anterior muscle of a lower leg associated with the foot is responsible for an inverted orientation of the foot. In some embodiments, the microprocessor 120 can determine that multiple muscles of the lower leg are attributable to an inverted orientation of the foot, such as, for example, the tibialis anterior muscle and the tibialis posterior muscle. The microprocessor 120 is configured to produce a output signal OP₄ based on the calculations and to control the stimulation of the attributable lower limb muscle via the stimulator unit 140.

The stimulator unit 140 (i.e., the muscle stimulator) is configured to receive the output signal OP₄ generated by the microprocessor 120 and to stimulate the muscle(s) identified by the microprocessor 120 via an electrode array (not shown). More specifically, the stimulator unit 140 is configured to generate a muscle stimulation signal based on the output signal OP₄ and send the muscle stimulation signal to the electrode array such that the electrode array performs the stimulation. The electrode array is coupled to the stimulator unit 140. The stimulator unit 140 is coupled to a lower limb of the body such that the electrode array is in contact with a portion of an outer surface of the skin of the lower limb. In this manner, the electrode array can deliver the stimulation via the outer surface of the skin (i.e., transcutaneously). The lower limb can be, for example, a lower leg associated with the foot. As a result of the stimulation, the orientation of the foot is corrected such that the gait returns to normal.

FIG. 2 is a perspective view of a system 200 according to an embodiment of the invention. The system 200 is configured to detect and correct an orientation of a foot of a body, such as, for example, an inversion or an eversion, during a gait cycle. The system 200 includes sensors 211, 212, and 213, a microprocessor 220 and a stimulator unit 240. The sensors 211, 212, and 213 are coupled to a foot F of a body B and arranged in a configuration such that the sensors 211 and 212 are coupled at the toe-end T of the foot F and sensor 213 is coupled at the heel-end H of the foot F. The configuration of the sensors 211, 212, and 213 on the foot F facilitates the detection of the orientation and the position of the foot F during a gait cycle. As such, the sensors 211, 212 and 213 can be arranged in any configuration that facilitates the detection of the orientation of the foot F, as discussed in more detail herein.

The sensors 211, 212, and 213 sense a foot-floor force reaction at the heel-end H and at the toe-end T of the foot F. More specifically, during a gait cycle, as shown in FIG. 2, the foot F moves in a heel H to toe T motion along a surface S of a floor such that a force is exerted on each sensor 211, 212, and 213. The sensors 211, 212, and 213 each generate an output signal OP₅, OP₆ and OP₇, respectively, associated with a threshold value corresponding to the amount of force exerted. In this manner, each of the output signals OP₅, OP₆ and OP₇ are associated with the orientation and/or the position of the foot F.

The sensors 211, 212, and 213 are coupled to the microprocessor 220 such that the microprocessor 220 receives the output signals OP₅, OP₆, and OP₇, generated by the sensors 211, 212, and 213. The microprocessor 220 can be, for example, an application-specific integrated circuit (ASIC) or a combination of ASICs, which are designed to perform one or more specific functions. In some embodiments, the microprocessor 220 can be an analog or digital circuit, or a combination of multiple circuits. The microprocessor 220 uses a pre-programmed algorithm to calculate the orientation and/or the position of the foot F. More specifically, the microprocessor 220 compares the threshold values corresponding with the output signals OP₅, OP₆ and OP₇ from the sensors 211, 212, and 213 to calculate the orientation of the foot F. For example, in use, the microprocessor 220 can compare sensor 211, associated with the inside of the foot F, with sensor 212, associated with the outside of the foot F, to determine if more force is being exerted on one than the other. If a disproportionate amount of force is applied to sensor 212 (i.e., the outside of the foot), for example, the microprocessor 220 will determine that the foot F is in an inverted orientation. In some embodiments, the microprocessor 220 can additionally calculate a position of the foot F during a gait cycle, as described in U.S. Patent Publication No. 2004-0122483 to Nathan et al., which is incorporated by reference for all purposes as if fully set forth herein.

When the microprocessor 220 calculates an abnormal orientation of the foot, as described above, the microprocessor 220 then determines the muscle attributable to the abnormal orientation. For example, the microprocessor 220 could determine that a tibialis anterior muscle of a lower leg associated with the foot is responsible for an inverted orientation of the foot. In some embodiments, the microprocessor 220 can determine that multiple muscles of the lower leg are attributable to an inverted orientation of the foot, such as, for example, the tibialis anterior muscle and the tibialis posterior muscle. The microprocessor 220 produces output signals OP₈, OP₉ and OP₁₀ based on the calculations and controls the stimulation of the attributable lower limb muscle via the stimulator unit 240.

The stimulator unit 240 is coupled to a lower limb L of the body B and receives the output signals OP₈, OP₉ and OP₁₀ generated by the microprocessor 220. The stimulator unit 240 includes an electrode array 250 having multiple electrodes (not shown) coupled to an outer surface of a skin of the lower limb L of the body B. Each electrode in the electrode array 250 is configured to stimulate a muscle of the lower limb L and is positioned on the skin to facilitate such stimulation. Additionally, each of the output signals OP₈, OP₉ and OP₁₀ is associated with an electrode from the electrode array 250 such that each output signal OP₈, OP₉ and OP₁₀ corresponds to the stimulation of a muscle of the lower limb L. Said another way, the microprocessor 220 controls the stimulation such that the microprocessor 220 can switch the stimulation between electrodes of the electrode array 250 via the output signals OP₈, OP₉ or OP₁₀. For example, a first electrode of the electrode array 250, corresponding to output signal OP₈, can be associated with a tibialis anterior muscle, responsible for an inverted orientation of the foot, such that the first electrode stimulates the tibialis anterior muscle to correct the inversion when the output signal OP₈ is received by the stimulator unit 240. Additionally, the output signals OP₈, OP₉ or OP₁₀ can correspond to the intensity of the stimulation. As a result of the stimulation, the abnormal orientation of the foot F is corrected.

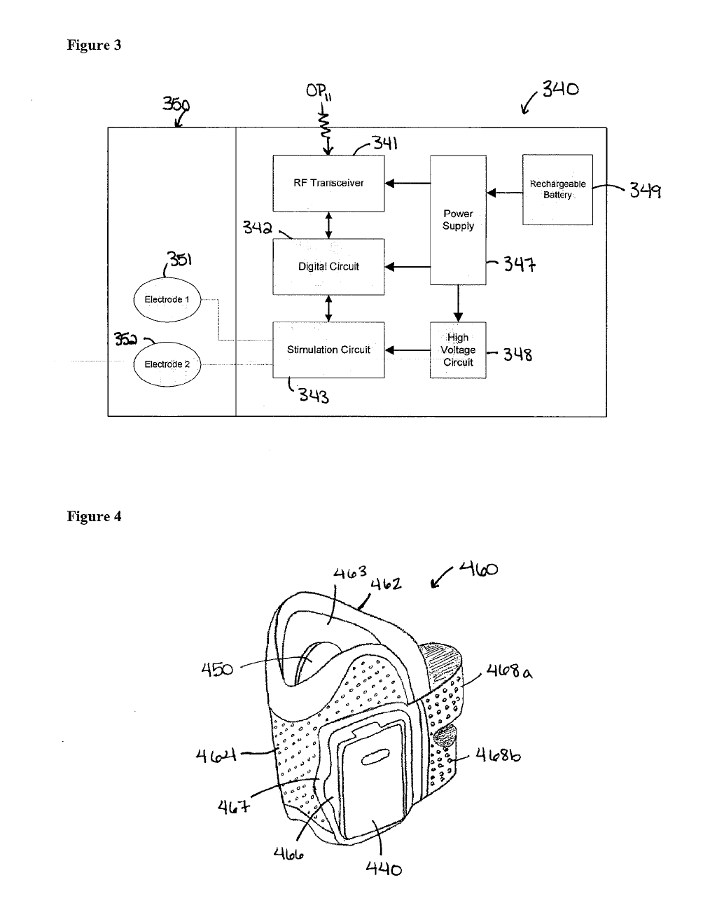

FIG. 3 is a block diagram of a stimulator unit 340 according to an embodiment of the invention. The stimulator unit 340 (i.e., muscle stimulator) is configured to be coupled to a lower limb of a body and provide electrical stimulation to a muscle. The stimulator unit 340 includes a radio frequency (RF) transceiver 341, a digital circuit 342, a stimulation circuit 343, a power supply 347, a high voltage circuit 348, a rechargeable battery 349, and an electrode array 350. The stimulator unit 340 is powered by the rechargeable battery 349 that is electrically coupled to the internal power supply 347. In this manner, the stimulator unit 340 can be used wirelessly. The power supply 347 supplies power to the RF transceiver 341, the digital circuit 342, and the high voltage circuit 348. The high voltage circuit 348 provides power to the stimulation circuit 343, which powers the electrode array 350. The stimulation circuit 343 is configured to generate a muscle stimulation signal and to send it to the electrode array 350. The electrode array 350 includes electrodes 351 and 352 which are electrically coupled to the stimulation circuit 343. The electrodes 351 and 352 are configured to be coupled to an outer surface of a skin of the lower limb of the body. Electrodes 351 and 352 of the electrode array 350 are each configured to stimulate a muscle of the lower limb based on the muscle stimulation signal and are positioned on the skin to facilitate such stimulation. In some embodiments, the electrode array 350 can include more than two electrodes.

The RF transceiver 341 is configured to receive an output signal OP₁₁ generated, for example, by a microprocessor. In some embodiments, the RF transceiver 341 can operate in the frequency range of 2400-2483.5 GHz. The digital circuit 342 provides an interface between the RF transceiver 341 and the stimulation circuit 343.

In use, the RF transceiver 341 receives an output signal OP₁₁ from, for example, a microprocessor. The output signal OP₁₁ contains information relating to the stimulation of a muscle, such as, for example, the electrode in the electrode array 350 associated with the muscle, the intensity of the stimulation, and/or the like. The output signal OP₁₁ is passed through the digital circuit 342 and to the stimulation circuit 343, which generates a muscle stimulation signal corresponding to the information from the output signal OP₁₁. Next, the stimulation circuit 343 sends the muscle stimulation signal to the appropriate electrode(s) 351 and/or 352 such that the electrode(s) 351 and/or 352 provide electrical stimulation to the body.

In some embodiments, a stimulator unit can be housed in an orthosis. For example, FIG. 4 is a perspective view of an orthosis 460 according to an embodiment of the invention. The orthosis 460 includes a frame 462, a handle 467, a cradle 466, a stimulator unit 440, an electrode array 450 and straps 468a and 468b. The frame 462, which can be constructed of, for example, a semi-rigid material, includes an internal layer 463 and an external layer 464. The frame 462 is coupled to the cradle 466 and the electrode array 450. The cradle 466 is configured to at least partially house the stimulator unit 440, which is electrically coupled to the cradle 466. The electrode array 450 is electrically coupled to the cradle 466 via the frame 462. In this manner, the electrode array 450 is electrically coupled to the stimulator unit 440.

The frame 462 is ergonomically configured to substantially envelop a lower limb of a body such that the orthosis 460 can be comfortably secured to the body. The orthosis 460 is secured to the body via the straps 468a and 468b and the handle 467. One end of the straps 468a and 468b is coupled to the frame 462 and the other end of the straps 468a and 468b is coupled to the handle 467. The handle 467 has a hollow rectangular shape that is configured to fit around the stimulator unit cradle 466, thereby securely locking the orthosis 460 into place over the lower limb.

By way of example, U.S. Patent Publication No. 2007-0112394 to Nathan et al., which is incorporated by reference for all purposes as if fully set forth herein, discloses such an orthosis housing a stimulator unit.

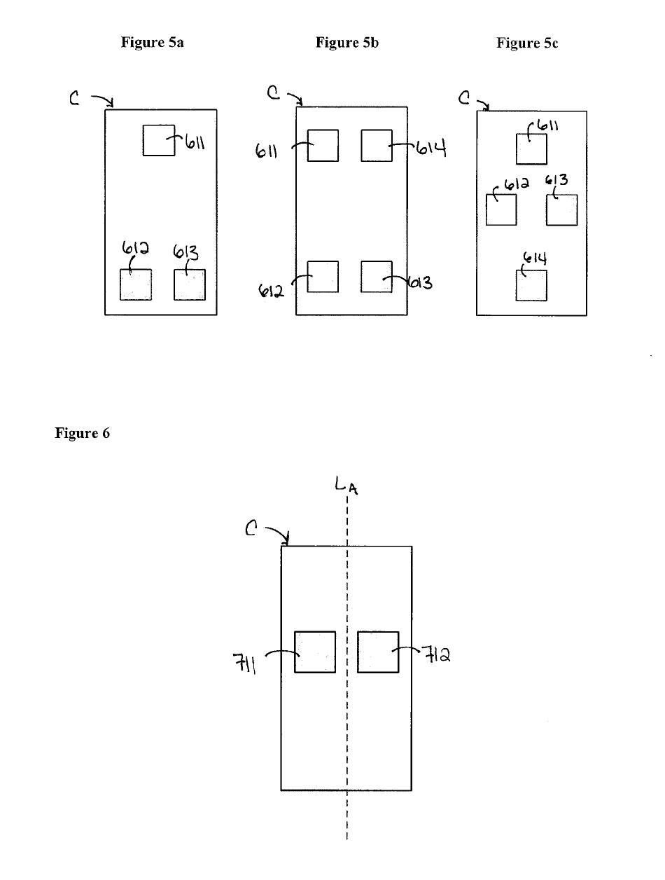

In some embodiments, as discussed above, a system can include sensors that are arranged in any configuration suitable to facilitate the detection of an orientation of a foot. In some embodiments, a system can include more than three sensors in a configuration. For example, FIGS. 5a-5c are schematic illustrations of sensor configurations according to embodiments of the invention. In FIG. 5a, sensors 611, 612 and 613 are arranged on a common plane C in a configuration opposite to that of the configuration illustrated in FIG. 1. An additional sensor, sensor 614, can be included in the system such that the system has four sensors 611, 612, 613, and 614 arranged in a configuration on the common plane C, as shown in FIG. 5b. Similarly, the sensors 611, 612, 613, and 614 can be configured on the common plane C, as shown in FIG. 5c, to facilitate the detection of the orientation of the foot. In some embodiments, the system can include more than four sensors.

In some embodiments, a system can include two sensors in a configuration suitable to facilitate the detection of an orientation of a foot. For example, FIG. 6 is a schematic illustration of sensors 711 and 712 arranged in a configuration on a common plane C. The sensors 711 and 712 are associated with a foot of a body. Sensor 711 is arranged on the left side of the longitudinal axis L_{A} and sensor 712 is arranged on the right side of the longitudinal axis L_{A}. Such a configuration enables the sensors 711 and 712 to facilitate the detection of an orientation of a foot of a body. More specifically, the sensors 711 and 712 show when an orientation is disproportionately to one side of the longitudinal axis L_{A} or the other. In this manner, the sensors 711 and 712 can sense when the foot is in an inverted or everted orientation. For example, when a right foot is in an inverted orientation during a gait cycle, a disproportionate amount of force will be applied to sensor 711 than to sensor 712.

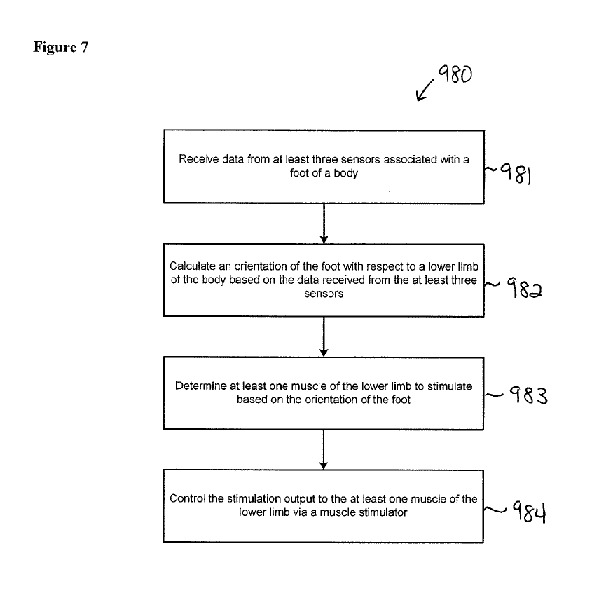

FIG. 7 is a flow chart of a method 980 for detecting and correcting an orientation of foot according to an embodiment of the invention. The method includes receiving data from at least three sensors associated with a foot of a body, 981. In some embodiments, the data is received by a microprocessor. The data can be, for example, associated with a force, a pressure, an angle, and/or an acceleration of a sensor.

The method includes calculating an orientation of the foot with respect to a lower limb of the body based on the data received from the at least three sensors, 982. The orientation can be, for example, an inversion or eversion of the foot. In some embodiments, the calculating the orientation of the foot includes comparing a parameter output, such as, for example, a force value, by each of the at least three sensors with a threshold value associated with each of the at least three sensors. In some embodiments, a position of the lower limb can be calculated based on the data received from the at least three sensors.

The method includes determining at least one muscle of the lower limb to stimulate based on the orientation of the foot, 983. In some embodiments, the determining occurs when the orientation of the foot is one of an inversion or an eversion.

The method includes controlling the stimulation output to the at least one muscle of the lower limb via a muscle stimulator, 984. In some embodiments, the controlling the stimulation output occurs when the orientation of the foot is one of an inversion or an eversion.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

In some embodiments, the sensors 111, 112, 113, 211, 212, and 213 can be wirelessly coupled to the microprocessor 120 and 220. In some embodiments, however, the sensors 111, 112, 113, 211, 212, and 213 can be coupled to the microprocessor 120 and 220 via a wire, cable and/or the like, as described in U.S. Patent Publication No. 2007-0112285 to Dar et al., which is incorporated by reference for all purposes as if fully set forth herein. Similarly, in some embodiments, the microprocessor 120 and 220 can be wirelessly coupled to the stimulator unit 140 and 240. In some embodiments, however, the microprocessor 120 and 220 can be coupled to the stimulator unit 140 and 240 via a wire, cable and/or the like.

In some embodiments, the output signals OP₁, OP₂, OP₃, OP₅, OP₆, and OP₇ of sensors 111, 112, 113, 211, 212, and 213 can be transmitted to the microprocessor 120, and 220 continuously. In some embodiments, however, the output signals OP₁, OP₂, OP₃, OP₅, OP₆, and OP₇ of sensors 111, 112, 113, 211, 212, and 213 can be generated as a result of sensors 111, 112, 113, 211, 212, and 213 being activated by a pressure, force, and/or the like, during a gate cycle. Similarly, in some embodiments, the output signal OP₄, OP₈, OP₉ and OP₁₀ can be transmitted to the stimulator unit 140 and 240 continuously. In some embodiments, however, the output signal OP₄, OP₈, OP₉ and OP₁₀ can be generated as a result of the sensors 111, 112, 113, 211, 212, and 213 being activated by a pressure, force, and/or the like, during a gate cycle.

In some embodiments, the microprocessor 120 and 220 can be configured to store the calculated orientation of the foot and data associated with the orientation such that the data can be downloaded to an external device, such as, for example, a personal computer.

Although the embodiments above describe a muscle stimulation using an external stimulation, in some embodiments, the electrode array 250, 350, and 450 can be implanted within a lower limb of the body and configured to stimulate the muscle directly.

Although various embodiments have been described as having particular features and/or combinations of components, other embodiments are possible having a combination of any features and/or components from any of the embodiments where appropriate.

What is claimed is:

1. A method, comprising:
receiving data from at least three sensors associated with a foot of a body;
calculating an orientation of the foot with respect to a lower limb of the body based on the data received from the at least three sensors;
determining at least one muscle of the lower limb to stimulate based on the orientation of the foot; and
controlling the stimulation output to the at least one muscle of the lower limb via a muscle stimulator.

2. The method of claim 1, wherein the orientation of the foot is one of an inversion or an eversion.

3. The method of claim 1, further comprising:
calculating a position of the lower limb based on the data received from the at least three sensors.

4. The method of claim 1, wherein the calculating the orientation of the foot includes comparing a parameter output by each of the at least three sensors with a threshold value associated with each of the at least three sensors.

5. The method of claim 1, wherein the controlling the stimulation output occurs when the orientation of the foot is one of an inversion or an eversion.

6. The method of claim 1, wherein the determining at least one muscle to stimulate occurs when the orientation of the foot is one of an inversion or an eversion.

7. A system, comprising:
at least three sensors configured to be arranged substantially in a common plane and associated with a foot of a body, each of the at least three sensors configured to produce a signal associated with an orientation of the foot;
an electrode array coupled to a lower limb of the body, the electrode array configured to stimulate at least one muscle of the lower limb;
a muscle stimulator coupled to the electrode array, the muscle stimulator configured to output a muscle stimulation signal to the electrode array; and
a microprocessor coupled to the at least three sensors, the microprocessor configured to calculate an orientation of the foot of the body based on the signals produced by the at least three sensors and to control the muscle stimulation output via the muscle stimulator based on the calculated orientation of the foot.

8. The system of claim 7, wherein the orientation of the foot is one of an inversion or an eversion.

9. The system of claim 7, wherein the electrode array is configured to contact at least a portion of an outer surface of the skin of the lower limb such that the array can deliver the stimulation via the outer surface of the skin.

10. The system of claim 7, wherein the at least three sensors includes one of an on-off switch, a force sensor, a pressure sensor, a gyroscopic tilt sensor, an accelerometer, or a goniometer.

11. The system of claim 7, wherein each of the at least three sensors outputs a signal associated with a threshold value.

12. The system of claim 7, wherein the microprocessor is configured to calculate the orientation of the foot by comparing the signals output by each of the at least three sensors with a threshold value associated with each of the at least three sensors.

13. The system of claim 7, wherein the microprocessor is configured to calculate a position of the lower limb based on the signals output by each of the at least three sensors.

14. The system of claim 7, wherein the microprocessor is configured to control the muscle stimulation output when the orientation of the foot is one of an inversion or an eversion.

15. The system of claim 7, wherein the stimulator is configured to output a muscle stimulation signal when the orientation of the foot is one of an inversion or an eversion.

16. A processor-readable medium storing code representing instructions to cause a processor to perform a process, the code comprising code to:
receive data from at least three sensors associated with a foot of a body;
calculate an orientation of the foot with respect to a lower limb of the body based on the data received from the at least three sensors;
determine at least one muscle of the lower limb to stimulate based on the orientation of the foot;
control the stimulation output to the at least one muscle of the lower limb via a muscle stimulator.

17. The processor-readable medium of claim 16, wherein the data received from the at least three sensors includes signals associated with the orientation of the foot, output by the at least three sensors.

18. The processor-readable medium of claim 16, further comprising:
calculate a position of the lower limb based on the data received from the at least three sensors.

19. The processor-readable medium of claim 16, wherein the control of the stimulation output occurs when the orientation is one of an inversion or an eversion.

20. The processor-readable medium of claim 16, wherein the calculating the position includes comparing signals associated with the orientation of the foot output by each of the at least three sensors with a threshold value associated with each of the at least three sensors.
